SEQUENCE LISTING (20 sequences)

SEQ ID NO: 1 - DNA, 20 bp, Artificial
  PCR primer for KCNMA1 and/or uPA gene
atatccgcccagacactgac

SEQ ID NO: 2 - DNA, 20 bp, Artificial
  PCR primer for KCNMA1 and/or uPA gene
atcgttggctgcaataaacc

SEQ ID NO: 3 - DNA, 20 bp, Artificial
  PCR primer for KCNMA1 and/or uPA gene
ttggaccaagacgatgatga

SEQ ID NO: 4 - DNA, 20 bp, Artificial
  PCR primer for KCNMA1 and/or uPA gene
cctctaagggcgttttcctc

SEQ ID NO: 5 - DNA, 20 bp, Artificial
  PCR primer for KCNMA1 and/or uPA gene
actccaaaggcagcaatgaa

SEQ ID NO: 6 - DNA, 20 bp, Artificial
  PCR primer for KCNMA1 and/or uPA gene
ggcctttcctcggtaaaagt

SEQ ID NO: 7 - DNA, 20 bp, Artificial
  PCR primer for KCNMA1 and/or uPA gene
gtcaccaccaaaatgctgtg

SEQ ID NO: 8 - DNA, 20 bp, Artificial
  PCR primer for KCNMA1 and/or uPA gene
gcggatccagggtaagaagt

SEQ ID NO: 9 - DNA, 21 bp, Artificial
  si RNA target sequence
gactggcagagtcctggttgt

SEQ ID NO: 10 - DNA, 21 bp, Artificial
  siRNA target sequence
gtgggtctgtccttccctact

SEQ ID NO: 11 - DNA, 21 bp, Artificial
  si RNA target sequence
gaccgtcctgagtggccatgt

SEQ ID NO: 12 - DNA, 21 bp, Artificial
  si RNA target sequence
acgcccttagaggtggctaca

SEQ ID NO: 13 - DNA, 19 bp, Artificial
  PCR Primer
ttctgggcctccttcgtct

SEQ ID NO: 14 - DNA, 18 bp, Artificial
  PCR primer
cctggcctcctccatggt

SEQ ID NO: 15 - DNA, 20 bp, Artificial
  PCR primer
accactacctgggcaaggag

SEQ ID NO: 16 - DNA, 19 bp, Artificial
  PCR primer
ttctgcatcacgtcccgga

SEQ ID NO: 17 - DNA, 20 bp, Artificial
  Hybridization probe
  misc_feature (20)..(20): fluorophore labeled thymine
agcgtccgccagagcaagan

SEQ ID NO: 18 - DNA, 23 bp, Artificial
  Hybridization probe
  misc_feature (23)..(23): phosphorylated thymine
atgaagaggcccccgaagaaagn

SEQ ID NO: 19 - DNA, 23 bp, Artificial
  Hybridization probe
  misc_feature (23)..(23): fluorophore labeled thymine
cagatggggccgaagatcctgtn

SEQ ID NO: 20 - DNA, 28 bp, Artificial
  Hybridization probe
  misc_feature (28)..(28): phosphorylated cytosine
caaatctcagcaccatgaggttctgcan

(19) United States
(12) Patent Application Publication
(10) Pub. No.: US 2007/0292528 A1
(43) Pub. Date: Dec. 20, 2007

(54) Kcnma1 as a Therapeutic Target in Cancer Treatment

(75) Inventors: Lukas Bubendorf, Therwil (CH); Michael Bloch, Oberwil (CH)

Correspondence Address: JOYCE VON NATZMER;PEQUIGNOT + MYERS LLC, 200 Madison Avenue, Suite 1901, New York, NY, 10016, US

(73) Assignee: University Hospital of Basel, Basel (CH)

(21) Appl. No.: 10/555,912

(22) PCT Filed: May 5, 2004
(86) PCT No.: PCT/EP04/04763

§ 371 Date: Nov. 2, 2006

Related U.S. Application Data
(60) Provisional application No. 60/468,056, filed on May 6, 2003.

(51) Int. Cl. A61K 35/56; A61P 35/00; C12Q 1/02; C12Q 1/68

(52) U.S. Cl. 424/537; 435/29; 435/6; 514/789

(57) ABSTRACT

The present invention pertains to a treatment of cancer, particularly prostate cancer, by blocking the large conductance, Ca²+-activated potassium channel KCNMA1 Embodiments of the invention include methods of detecting the level of expression of KCNMA1, methods for treating patients with prostate cancer, and methods of discovering drugs for treating cancer.

### BACKGROUND OF THE INVENTION

1. Field of the Invention

The present invention pertains to treatment and of cancer, particularly prostate cancer, by targeting KCNMA1.

2. Background of the Invention

Prostate cancer is the most frequent malignant tumor among males in western countries and the second leading cause of cancer-related death. Most primary prostate cancers initially respond favorably to androgen withdrawal therapy. However, they almost invariably recur as hormone-refractory tumors after several months to a few years. No effective therapies currently exist for end-stage hormone-refractory and metastatic prostate cancer. It would therefore be important to better understand the biological basis of prostate cancer progression in order to identify new therapeutic avenues. Since cancer is based, at least in part, on genetic alterations, the detection of chromosomal changes can pinpoint critical genes and highlight mechanisms of cancer development and progression. Studies by comparative genomic hybridization (CGH) carried out at our laboratory suggest that the chromosomal region 10q22 contains one or several oncogenes with relevance for the progression to late stage prostate cancer (El Gedaily, A. et al.: Discovery of new amplification loci in prostate cancer by comparative genomic hybridization. Prostate 2001, 46:184-190). This hypothesis is based on the observation that 10q22 amplification is present in the hormone-refractory prostate cancer cell lines PC-3, and also in 10% of hormone-refractory human prostate cancers. DNA amplification sites can harbor potent oncogenes that drive tumor progression and might qualify as therapeutic targets. For example, metastatic breast cancers with amplification of the HER2/neu gene respond to treatment with trastuzumab (Herceptin™), a therapeutic antibody that is directed against the Her-2 protein. The Ca²⁺-activated large conductance K⁺-channel (KCNMA1; BK channel) is one of the genes of the 10q22 amplification site in prostate cancer. KCNMA1 encodes for the pore-forming α-subunit of the channel, while the four regulatory B-subunits are encoded by KCNMB1-4. KCNMA1 is a key modulator of vascular smooth muscle tone and plays a role in synaptic neurotransmitter release. Hence, potassium channel modulating agents have been suggested as therapeutic agents in neurologic and cardiovascular disorder (Calderone, V: Large-conductance, ca(2+)-activated k(+) channels: function, pharmacology and drugs. Curr Med Chem 2002, 9:1385-95). In addition, it has been suggested that potassium channels may be involved in oncogenesis. For example, KCNMA1 activation has been shown to drive tumor cell proliferation in astrocytoma (Basrai, D, et al.: BK channel blockers inhibit potassium-induced proliferation of human astrocytoma cells. Neuroreport 2002, 13:403-7). It has also been recognized that potassium channel may play a role in the progression of prostate cancer (Abdul, M, Hoosein, N: Expression and activity of potassium ion channels in human prostate cancer. Cancer Lett 2002, 186:99-105). However, the potassium channel KCNMA1 has not been previously analyzed in prostate cancer. Notably, an oncogenic potential of potassium channels is also emphasized by the recently reported amplification and overexpression of KCNK9 at 18q24 in 10% of breast cancers (Mu, D., et al.: Genomic amplification and oncogenic properties of the KCNK9 potassium channel gene. Cancer Cell 2003; 3:297-302).

The present invention relates to our findings regarding the biological significance of KCNMA1 amplification in prostate cancer and its potential as a new therapeutic target.

High-level amplifications, which represent narrow chromosomal regions with a highly increased copy number of DNA sequences, often lead to a dramatic overexpression of genes within the amplified region. Amplified and overexpressed genes can result in a growth advantage of affected tumor cell clones that eventually determine the biological behavior of the tumor. High-level gene amplifications are rare in primary prostate cancer, but have been reported in advanced tumors. The most frequent high-level amplification in prostate cancer was found at XQ11.2-12 (El Gedaily, A. et al.: Discovery of new amplification loci in prostate cancer by comparative genomic hybridization. Prostate 2001, 46:184-190). This amplification is present in 20-30% of hormone-refractory prostate cancers and has never been described in any other tumor type than prostate cancer. The androgen receptor (AR) gene is the most likely target of this amplification by FISH (Bubendorf, L., et al.: Survey of gene amplifications during prostate cancer progression by high-throughput fluorescence in situ hybridisation on tissue microarrays. Cancer Res 1999, 59:803-806). AR amplified tumor cells may become hypersensitive to the remaining low levels of androgen after androgen withdrawal and thereby retain AR-mediated growth signaling. Accordingly, AR-amplified hormone-refractory prostate cancers have been shown to better respond to second-line total androgen blockage than tumors without AR amplification. Other amplifications are less frequent in prostate cancer. However, amplified genes detected in only a small fraction of tumors or in individual tumors may be overexpressed in a much larger fraction of tumors through alternative mechanisms of activation (e.g. mutation, translocation, or posttranslational activation). Even oncogenes that are overexpressed in a small fraction of patients may be clinically relevant, if they can be used as a target for new efficient therapies.

### SUMMARY OF THE INVENTION

A first preferred embodiment of the invention is a method for detecting the expression level of KCNMA1, and optionally Urokinase (uPA), in a tissue sample, the method comprising the steps of: providing one or more probes and a tissue sample, the tissue sample including nucleotides; hybridizing the one or more probes to the nucleotides in the tissue sample to produce hybridization results; and determining an expression level for the KCNMA1 gene, and, optionally for the Urokinase (uPA) gene, from the hybridization results.

In a further preferred embodiment, the hybridization of the first embodiment comprises in situ hybridization.

In yet another preferred embodiment, the hybridization of the first embodiment occurs in a tissue microarray.

Still another preferred embodiment, the first embodiment further comprises a step of amplifying the KCNMA1 gene, and optionally the Urokinase (uPA) gene, from the tissue to produce amplified DNA.

In a still further preferred embodiment, the first embodiment further comprises the step of identifying a level of expression of KCNMA1 mRNA, and, optionally, the Urokinase (uPA) gene, by using a primer selected from the group consisting of: KCNMA1f, KCNMA1r, and KCNMA1 probes.

In yet another preferred embodiment, the tissue sample collected in the first embodiment is a prostate tumor tissue sample.

In yet another preferred embodiment, the first embodiment further comprises the step of identifying a KCNMA1 mRNA, and optionally a Urokinase (uPA) mRNA, by using one or more PCR primers selected from the group consisting of KCNMA1f: ATATCCGCCCAGACACTGAC, KCNMA1r: ATCGTTGGCTGCAATAAACC, KCNMA2f:
TTGGACCAAGACGATGATGA, KCNMA2r: CCTCTAAGGGCGTTTTCCTC, Plau1f: ACTCCAAAGGCAGCAATGAA, Plau1r:
GGCCTTTCCTCGGTAAAAGT, Plau2f: GTCACCACCAAAATGCTGTG, and Plau2R: GCGGATCCAGGGTAAGAAGT.

A second preferred embodiment is a method for treating a patient with cancer comprising the steps of: collecting a tumor tissue sample; analyzing the tumor tissue sample to determine an expression level of KCNMA1, and optionally Urokinase (uPA), to determine whether KCNMA1 overexpression is absent or present and whether Urokinase (uPA) overexpression is present or absent; and when KCNMA1 overexpression or Urokinase (uPA) overexpression is present, treating the patient with an anti-tumor agent

In another preferred embodiment, the anti-tumor agent of the second embodiment is a blocking agent of potassium channels.

In a still further preferred embodiment, the blocking agent of the second embodiment is iberiotoxin.

In a third preferred embodiment, the patient of the second embodiment is treated with the anti-tumor agent when both KCNMA1 gene amplification and Urokinase (uPA) gene amplification are present.

In another preferred embodiment, the anti-tumor agent of the third embodiment is a blocking agent of potassium channels.

In a still further preferred embodiment, the blocking agent of the third embodiment is iberiotoxin.

In a yet another preferred embodiment, the second embodiment further comprises the step of identifying the KCNMA1 gene, and optionally the Urokinase (uPA) gene, by using one or more PCR primers selected from the group consisting of KCNMA1f: ATATCCGCCCAGACACTGAC, KCNMA1r: ATCGTTGGCTGCAATAAACC, KCNMA2f:
TTGGACCAAGACGATGATGA, KCNMA2r: CCTCTAAGGGCGTTTTCCTC, Plau1f: ACTCCAAAGGCAGCAATGAA, Plau1r:
GGCCTTTCCTCGGTAAAAGT, Plau2f: GTCACCACCAAAATGCTGTG, and Plau2R: GCGGATCCAGGGTAAGAAGT.

In a fourth embodiment of the invention, the cancer of the second embodiment is prostate cancer.

In yet another embodiment, the patient of the fourth embodiment has developed, is developing, or is suspected to have developed hormone-refractory prostate cancer.

A fifth preferred embodiment is a method of identifying a drug for treating cancer, comprising the steps of: providing a first cell line expressing a large-conductance, Ca2+-activated potassium channel; providing a second cell line which expresses the channel at a lower level than the first cell line, or does not express the channel at all; providing a candidate compound for treating cancer; incubating the candidate compound with the cell lines to produce a response in each cell line; comparing the response of the first cell line to the response of the second cell line.

A still further preferred embodiment, the candidate compound of the fifth embodiment comprises a compound known or suspected to block a potassium channel.

In yet another preferred embodiment, the response of the fifth embodiment is a rate of growth of the cell line.

In still another preferred embodiment, the channel of the fifth embodiment is KCNMA1.

Yet another preferred embodiment is a compound identified by the method of the fifth embodiment.

Further objects features and advantages of the present invention will become apparent from the Detailed Description of the Invention, which follows, when considered together with the attached drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows CGH profiles of tumors and the PC-3 cell line with increased DNA sequence copy numbers at 10q22. CGH profiles are shown next to ideograms of chromosomes 10. In all tumors circumscribed peaks are visible at 10q22 indicating high-level amplification (PC-3 and patients 1-2) or gain (patients 3-4) of this region.

FIG. 2 shows a prostate cancer paraffin embedded tissue microarray with 535 specimens from 470 tumors and 65 benign controls with Hematoxylin-Eosin-staining (“H&E staining”).

FIG. 3 shows a frozen multi-tumor tissue microarray containing frozen specimens for mRNA-ISH: (a) a frozen TMA block; (b) a TMA specimen showing colorectal cancer with H&E staining; (c) an overview of frozen TMA with H&E staining; (d) mRNA ISH using a radioactively labeled cDNA probe; (e) an automated quantification of radioactive hybridization signals.

FIG. 4 shows (A) a 7 MB spanning map of the 10q22 amplicon core. Evaluated FISH probes (BAC) are indicated in relation to the genes they contain sequences of. Note: the alignment software was not able to reconstruct one single contig, despite the BAC overlap. Source: www.ensembl.org (B) An amplicon map of the 10q22 core amplicon in PC-3. The amplification ratio=average signals (probe)/average signals (centromer) is shown.

FIG. 5 shows gene expression relative to PC-3. Normalized gene expression of growing cell lines LNCaP, CWR22R, and BPH-1 were compared to the cell line PC-3 using LightCycler technology. Positive values indicate an increased expression level in PC-3 whereas negative values indicate a reduced expression level in PC-3. The mRNA expression levels were normalized to Beta-actin as a housekeeping gene. mRNA was extracted using the RNAeasy kit (Invitrogen) and transcribed into sscDNA with oligo dT Primers using the Superscript First Strand Synthesis System for RT PCR (Invitrogen). RT PCR was performed using a LightCycler device (Roche Molecular Diagnostics) and the LightCycler Fast Start DNA Master Sybr Green kit (Roche Molecular Diagnostics).

FIG. 6 shows FISH analysis of KCNMA1 amplification. An amplification of KCNMA1 genomic sequences was observed in 13.5% of advanced prostate tumors. But so far this amplification was never seen in earlier stages of this disease. Amplification was defined as a ratio of probe (green spots) versus centromer (red spots) of greater than 2.5. Centromer Probe: Cen10 spectrum orange (Vysis) KCNMA1 probe: Digoxigenin labeled Bac RP11-428P16, blue: nucleus. (A) normal cell (B) clinical tumor (C) PC-3 cell.

FIG. 7 shows the effect of iberiotoxin and estradiol on the cell lines PC-3 (A), LNCaP (B), and BPH-1(C). “K” represents a control; “I” represents treatment with 0.05 μM Iberiotoxin (Sigma); “E” represents treatment with 0.1 μM Estradiol; and “E+I” represents treatment with both 0.05 μM Iberiotoxin and 0.1 μM Estradiol. Cells were grown in OPTI-MEM (Invitrogen) +0.4% Albu-max (Invitrogen) for two days. It is demonstrated that Iberiotoxin reduces the growth rate of PC-3 by 10-15% whereas no effect is seen for the cell lines LNCaP and BPH-1. Iberiotoxin blocked the growth stimulus of Estradiol to the cell lines LNCAP and BPH-1.

FIG. 8 shows: (A) RT-PCR analysis of KCNMA1 expression one day after anti KCNMA1 siRNA treatment in PC-3. The siRNAe reduce KCNMA1 expression by 2-4 PCR cycles, which corresponds to an estimated downregulation of 70-90%. RT PCR was performed in a LightCycler device (Roche Molecular Diagnostics) using Hybridisation probes (TIB-Molbiol). (B) Growth of PC-3 treated with anti KCNMA1 siRNA. In the controls there is continuous growth, whereas the cells treated with siRNA showed a dramatic growth reduction.

FIG. 9 shows phenotypes of PC-3 cells 4 days after siRNA transfection. The cells become more spread, rather rectangular shaped when transfected with the siRNA K1, K2, and K4. In contrast to this phenotype, the cells shift to a elongated shape with reduced diameter when treated with the siRNA K3. “CO” represents no transfection; “c-si” represents only transfection agent; “scr” represents transfection of a scrambled sequence control siRNA (Qiagen); and “K1-4” represents transfection of individual anti KCNMA1 siRNA (Qiagen).

FIG. 10 shows patch clamp recordings of K⁺ channels present in cell excised inside/out membrane patches of PC-3 (A) and BPH-1 (B) cells. Numerous channels are active under high (1 mmol/l) intracellular Ca²⁺ concentration in the membrane patch from PC-3 cells (A), thus single channel currents cannot be resolved. In contrast, little channel activity is present in the BPH-1 membrane patch (B).

### DETAILED DESCRIPTION OF THE INVENTION

The term “overexpression” as used herein and in the appended claims refers to overexpression of a gene or gene product, such gene products including mRNA, other intermediate nucleotides, and protein, unless the term “overexpression” is used in a context clearly limited to a subset of the above.

The present inventors have found that KCNMA1 is amplified and overexpressed in the hormone-insensitive prostate cancer cell line PC-3, but not in the hormone-sensitive cell line LNCAP and in the benign control cell line BPH-1. Moreover, KCNMA1 overexpression appears to drive tumor cell proliferation in PC-3 as evidenced by the fact that iberiotoxin, a specific KCNMA1-blocker, leads to a significantly decreased growth rate. Also, siRNA against KCNMA1 resulted in a dramatic growth reduction of PC-3. Importantly, FISH analysis on a prostate cancer tissue microarray using a KCNMA1 specific BAC probe revealed that KCNMA1 amplification is not restricted to the in vitro model system PC-3, but also prevails in 10-15% of human hormone-refractory prostate cancers in vivo.

There is evidence that the activity of KCNMA1 may be influenced by several other proteins such as 17β-estradiol and urokinase (Valverde, M. A., et al.: Acute activation of Maxi-K channels (hSlo) by estradiol binding to the beta subunit. Science 1999, 285:1929-31; and Christow, S. P., et al.: Urokinase activates calcium-dependent potassium channels in U937 cells via calcium release from intracellular stores. Eur J Biochem 1999, 265:264-72.). The present inventors have found that an increased proliferation of LNCaP and BPH-1 induced by 17β-estradiol was reversed by the KCNMA1 blocker iberiotoxin, suggesting that 17β-estradiol drives tumor cell proliferation of LNCaP through activation of KCNMA1. This is in agreement with previous data showing that KCNMA1 activity in aortic smooth muscle cells is modulated by 17β-estradiol through its regulatory β-subunit (Valverde, M. A., et al.: Acute activation of Maxi-K channels (hSlo) by estradiol binding to the beta subunit. Science 1999, 285:1929-31). Similarly, tamoxifen, a chemotherapeutic xenoestrogen used for the treatment of patients with estrogen-receptor positive breast cancer, has been shown to increase the activity of BK channels in canine colonic myocytes. KCNMA1 has also been suggested to be under hormonal control in the myometrium. Other than in LNCaP and BPH-1, we detected no effect of 17β-estradiol on the growth of PC-3. It is believed that 17β-estradiol has no measurable effect on KCNMA1 in PC-3, since hyperactivity of KCNMA1 is already achieved through overexpression due to amplification.

Urokinase (uPA) has been shown to activate calcium-activated potassium channels in the human promyelocytic cell line U937 via calcium release from intracellular stores (Christow, S. P., et al.: Urokinase activates calcium-dependent potassium channels in U937 cells via calcium release from intracellular stores. Eur J Biochem 1999, 265:264-72.). Interestingly, uPA colocates with KCNMA1 to 10q22 and has recently been suggested as a target gene of this amplification site in prostate cancer (Helenius, M. A. et al.: Amplification of urokinase gene in prostate cancer. Cancer Res 2001, 61:5340-4). In fact, the present inventors have found that uPA was highly expressed and amplified at a similar level as KCNMA1 in PC-3. This suggests that the growth promoting effect of the KCNMA1 amplification is potentiated by the simultaneous amplification of its activator uPA. This hypothesis is in agreement with previous data indicating that functionally related genes tend to colocalize in the genome, and that DNA amplification sites may contain several critical target genes rather than one single target. However, in contrast to KCNMA1, high uPA expression was not limited to PC-3 in our real-time PCR analysis, but was also present in the control cell line BPH-1 from benign prostate. Therefore uPA overexpression may not be strictly malignancy-associated but also exert physiological functions in the benign prostate.

In conclusion, the present inventors have found that KCNMA1 enhances the proliferation of the PC-3 prostate cancer cell line through amplification and overexpression in vitro, and may also contribute to an aggressive and hormone-refractory growth in a fraction of clinical prostate cancers in vivo. Inhibition of KCNMA1 using specific channel inhibitors reveals a new targeted therapeutic strategy in patients suffering from advanced prostate cancer or other tumor types with KCNMA1 amplification and overexpression.

Identification of KCNMA1 as a Target in Treating Prostate Cancer

The present inventors have discovered that the potassium channel KCNMA1 is the target gene of the 10q22 amplification seen in prostate cancer. Quantitative real-time RT-PCR revealed a consistent association between KCNMA1 amplification and overexpression in the PC-3 cell line. KCNMA1 is not only amplified in the PC-3 cell line, but also in 13.5% of hormone-refractory local recurrences and metastatic deposits of clinical prostate cancers. siRNA against KCNMA1 resulted in a dramatic growth reduction of PC-3. Most importantly, specific blocking by the specific KCNMA1 inhibitor iberiotoxin also lead to a significant reduction of the growth rate in PC-3, but not in the non-amplified cell lines BPH-1 and LNCaP. These experiments not only demonstrate a role of KCNMA1 for prostate cancer cell growth, but also show that the gene is a useful drug target. KCNMA1 has diverse known functions, including modulation of smooth muscle tone, regulation of arterial blood pressure, and synaptic neurotransmitter release. Other studies that links KCNMA1 activation to the regulation of tumor cell proliferation in astrocytoma (Basrai D, Kraft R, Bollensdorff C, Liebmann L, Benndorf K, Patt S. BK channel blockers inhibit potassium-induced proliferation of human astrocytoma cells. Neuroreport 2002; 13:403-7) provide strong additional evidence for a role of this gene in cancer biology. An oncogenic potential of potassium channels is also emphasized by the recently reported amplification and overexpression of KCNK9 at 18q24 in 10% of breast cancers.

The molecular biology of prostate cancer has been a focus of our research for several years. Initially the present inventors explored new prognostic markers in a series of 137 radical prostatectomy specimens of patients with clinically localized prostate cancer (Bubendorf L., et al. Ki67 labeling index: an independent predictor of progression in prostate cancer treated by radical prostatectomy. J Pathol 1996;

178:437-41; and Bubendorf L., et al. Prognostic significance of Bcl-2 in clinically localized prostate cancer. Am J Pathol 1996; 148:1557-65). Immunohistochemical analysis revealed that the tumor growth fraction measured by the Ki67 Labeling Index (LI), as well as p53 and Bcl-2 expression were predictors of progression, and Ki67 LI emerged as an independent prognostic factor. New prognostic factors are even more warranted in core needle biopsies than in radical prostatectomy specimens, since the critical therapy decisions are made at the time of the initial core needle biopsies both in patients with clinically localized (and hence potentially curable) as well as in patients with advanced disease. Therefore, we sought to explore the significance of molecular markers in core needle biopsies. Ki67 LI was again an independent prognostic factor, supporting its potential as an adjunct to routine diagnostics in prostate cancer (Bubendorf L., et al. Ki67 Labeling Index in core needle biopsies independently predicts tumor-specific survival in prostate cancer. Hum Pathol 1998; 29:949-954). There has been continuous interest in the concept and biological significance of focal neuroendocrine differentiation in prostate cancer (Abrahamsson P A., Neuroendocrine differentiation in prostatic carcinoma. Prostate 1999;

39:135-48). As others, we did not find any prognostic significance of neuroendocrine differentiation in prostate cancer (Bubendorf L., et al. Ki67 labeling index: an independent predictor of progression in prostate cancer treated by radical prostatectomy. J Pathol 1996; 178:437-41; and Casella, R., et al. Focal neuroendocrine differentiation lacks prognostic significance in prostate core needle biopsies. J Urol 1998; 160:406-10). However, focal neuroendocrine differentiation appeared to be more frequent in hormone-refractory recurrences and metastases than in primary untreated tumors (Casella, R., et al. Focal neuroendocrine differentiation lacks prognostic significance in prostate core needle biopsies. J Urol 1998; 160:406-10). Thus, focal NE differentiation might be involved in hormone-refractory growth, possibly by paracrine stimulation of tumor cell proliferation or angiogenesis (Abrahamsson, P. A. Neuroendocrine differentiation in pro static carcinoma. Prostate 1999; 39:135-48). Using tissue microarray technology, the present inventors confirmed the prognostic significance of Ki67 LI in a series of >500 patients with long-term follow-up after radical prostatectomy, and found a prognostic role of Syndecan-1 (CD 138) in prostate cancer (Zellweger T., et al. Tissue microarray analysis reveals prognostic significance of syndecan-1 expression in prostate cancer. Prostate 2003; 55:20-9). In addition, a response of Ki67, Bcl-2 and CD10 expression to neoadjuvant hormonal treatment was demonstrated (Zellweger T., et al. Tissue microarray analysis reveals prognostic significance of syndecan-1 expression in prostate cancer. Prostate 2003; 55:20-9). In a subsequent analysis, the present inventors applied immunohistochemistry to a prostate progression tissue microarray (TMA) to explore the expression of 11 potential therapeutic targets across the whole spectrum of prostate cancer progression. It was found that p53, Bcl-2; Syndecan-1, EGFR and HER2/neu are preferentially expressed in hormone-refractory and metastatic prostate cancer. In conclusion, molecular markers add substantial prognostic information even in small biopsies of prostate cancer, and help to predict the response to targeted therapy.

Since molecular alterations in cancer often result from chromosomal alterations, the present inventors used CGH to explore the chromosomal aberrations that occur during the progression of prostate cancer (Fu W, Bubendorf L, Willi N, et al. Genetic changes in clinically organ-confined prostate cancer by comparative genomic hybridization. Urology 2000; 56:880-5; and ElGedaily A, Bubendorf L, Willi N, et al. Discovery of new amplification loci in prostate cancer by comparative genomic hybridization. Prostate 2001; 46:184-190). Different stages of progression were analyzed, including 28 tumors that were still organ-confined at the time of radical prostatectomy (stage pT2), 28 tumors with infiltration of the seminal vesicle (pT3b), and 27 advanced, mostly hormone-refractory tumors. Most of the chromosomal changes found in our studies have previously been described in prostate cancer, but were not systematically analyzed across different stages of tumor progression. Several chromosomal changes were significantly more frequent in the 27 advanced tumors as compared to the 56 clinically localized tumors. They included loss of 6q, 8p, 10q, 13q, 16q, and 18q, and gain of 8q, suggesting that genes with a role in prostate cancer progression are located on these chromosomal arms (Table 1).

TABLE 1
Significant chromosomal alterations during the progression of
prostate cancer

| | n= | 6q− | 8p− | 8q+ | 13q− | 16q− | 18q− |
|---|---|---|---|---|---|---|---|
| pT2 | 28 | 14% | 11% | 0% | 21% | 0% | 4% |
| pT3 | 28 | 14% | 32% | 18% | 21% | 4% | 21% |
| Hr recur* | 27 | 48% | 52% | 48% | 52% | 26% | 37% |
| p-value | | 0.004 | 0.0045 | 0.0001 | 0.02 | 0.002 | 0.009 |

*hormone-refractory local recurrence

The consistent finding of specific chromosomal alterations in prostate cancer suggests that they do not occur randomly, but may rather reflect activation or suppression of genes involved in tumor progression. Importantly, it was also found sixteen high-level amplifications in the group of advanced tumors (ElGedaily A, Bubendorf L, Willi N, et al. Discovery of new amplification loci in prostate cancer by comparative genomic hybridization. Prostate 2001; 46:184-190). These included Xq12 (five), 8q24 (two) and 11q13 (one) with known putative target genes (androgen receptor, MYC and PSMA, and Cyclin D1).

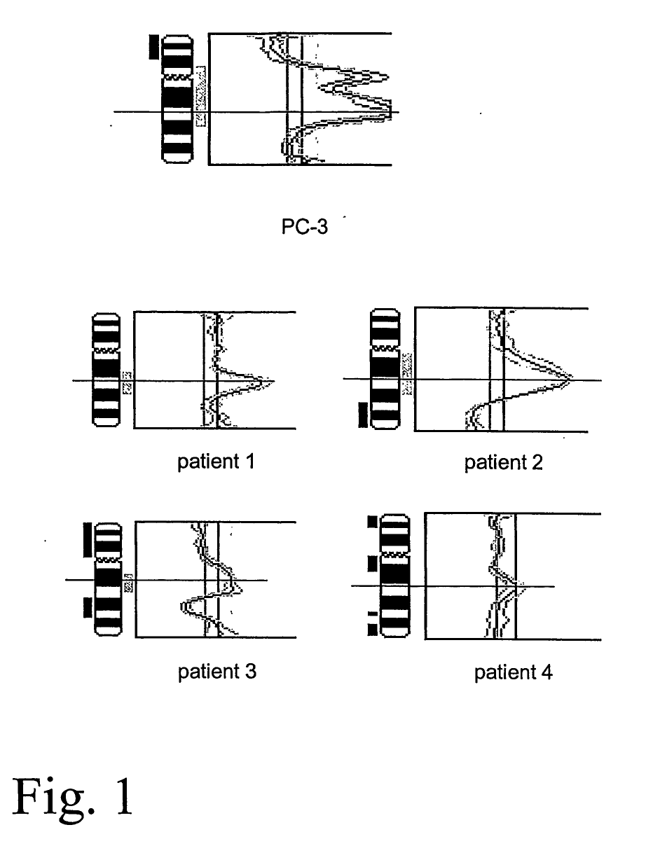

The most significant finding were high-level amplifications at 1q21-25 (3/27 tumors), 10q22, 17q24 (2/27 tumors, each), and 8q21 (one tumor). The target genes at these amplification loci are largely unknown. There are many examples in the literature that the identification of new amplifications can serve as a first step for the subsequent identification of biologically meaningful oncogenes (Knuutila S, Bjorkqvist A M, Autio K, et al. DNA copy number amplifications in human neo-plasms: review of comparative genomic hybridization studies. Am J Pathol 1998; 152:1107-23; Visakorpi T, Hyytinen E, Koivisto P, et al. In vivo amplification of the androgen receptor gene and progression of human prostate cancer. Nat Genet 1995; 9:401-6; and Barlund M, Forozan F, Kononen J, et al. Detecting activation of ribosomal protein S6 kinase by complementary DNA and tissue microarray analysis. J Natl Cancer Inst 2000; 92:1252-1259). Among these amplification sites, the 10q22 region has attracted our main interest because it is also present in the prostate cancer cell line PC-3 (FIG. 1 and Bernardino J, Bourgeois C A, Muleris M, Dutrillaux A M, Malfoy B, Dutrillaux B. Characterization of chromosome changes in two human prostatic carcinoma cell lines (PC-3 and DU145) using chromosome painting and comparative genomic hybridization (Cancer Genet Cytogenet 1997; 96:123-8; and Pan Y, Lui WO, Nupponen N, et al. 5q11, 8p11, and 10q22 are recurrent chromosomal breakpoints in prostate cancer cell lines. Genes Chromosomes Cancer 2001; 30:187-195). This cell line is a renewable resource that can be utilized as a model system in functional assays and DNA microarray analyses. Amplification at 10q22 has only exceptionally been reported in tumors other than prostate cancer (http://www.helsinki.fi/cmg). Amplification prevalence at 10q22 in 33% of metastatic bladder cancers was reported by one group, but not yet confirmed by others (Hovey R M, Chu L, Balazs M, et al. Genetic alterations in primary bladder cancers and their metastases. Cancer Res 1998; 58:3555-60). Notably, 10q22 amplification was identified by CGH in the non-small cell lung cancer cell line 1262T (Taguchi T, Cheng G Z, Bell D W, et al. Combined chromosome microdissection and comparative genomic hybridization detect multiple sites of amplification DNA in a human lung carcinoma cell line. Genes Chromosomes Cancer 1997; 20:208-12), but has not yet been described in clinical lung cancer.

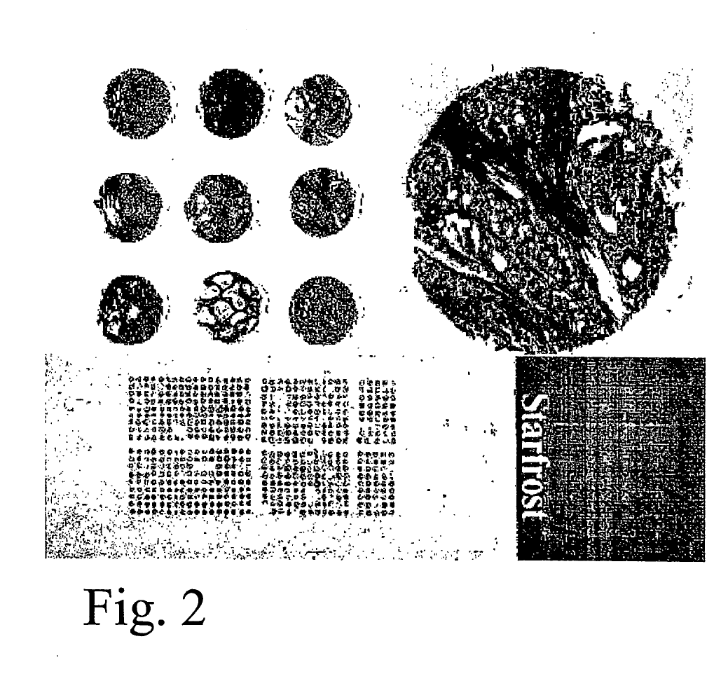

The present inventors developed the tissue microarray (TMA) technology together with the group of Olli Kallioniemi at NHGRI, Bethesda (FIG. 2 and Kononen J, Bubendorf L, Kallioniemi A, et al. Tissue microarrays for high-throughput molecular profiling of tumor specimens. Nat Med 1998; 4:844-7; and Bubendorf L, Nocito A, Moch H, Sauter G. Tissue microarray (TMA) technology: miniaturized pathology archives for high-throughput in situ studies. J Pathol 2001; 195:72-9). Since then, the inventors have constructed a large number of different tissue microarrays of many different tumor types, including multi-tumor TMAs, normal tissue TMAs, lung, breast, colorectal, ovarian, and prostate cancer TMAs (Bubendorf L, Nocito A, Moch H, Sauter G. Tissue microarray (TMA) technology: miniaturized pathology archives for high-throughput in situ studies. J Pathol 2001; 195:72-9). The prostate TMA's also include a sizeable number of distant metastases. These were mainly selected from an autopsy study on 1,589 patients, where we could demonstrate strong evidence for the importance of different metastatic pathways in prostate cancer (Bubendorf L, Schopfer A, Wagner U, et al. Metastatic patterns of prostate cancer: an autopsy study of 1,589 patients. Hum Pathol 2000; 31:578-83). The availability of high numbers of tumors and the capability to analyze candidate genes for amplification and expression in a high number of tumors is an important prerequisite for a successful exploration of potential amplification targets. We have the conditions and high experience to meet these requirements. In addition, we have collected fresh tissue from patients undergoing palliative transurethral resection for hormone-refractory local recurrence to be able to perform analyses that require fresh frozen material.

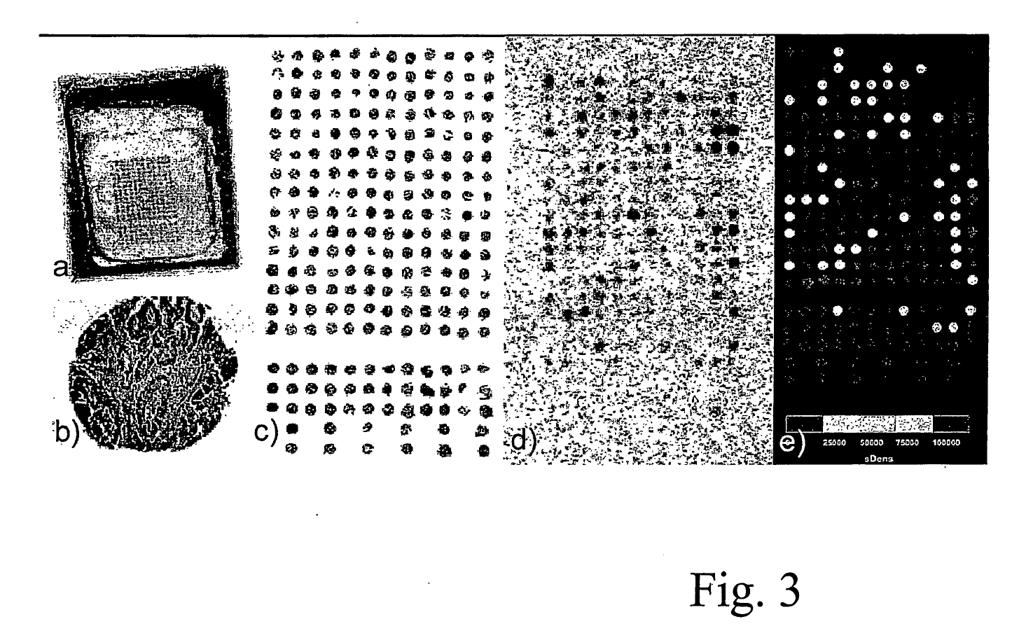

Since antibodies that work on formalin-fixed and paraffin-embedded tissues are available for only a small fraction of the known genes, mRNA in situ hybridization (mRNA ISH) is critical for the expression analysis of new candidate genes EST's. However, mRNA ISH on archival tumor material is problematic because of the degradation and cross-linking of RNA molecules by formalin fixation. Therefore, “frozen tissue microarrays” for mRNA ISH have been developed at our institute. “Frozen tissue microarrays” contain samples from deep frozen tissues that have been arrayed in a frozen state into a frozen recipient block (FIG. 3). mRNA ISH can be successfully performed on frozen sections of these arrays to survey the expression of genes and EST's, for which antibodies are not available. We have the following two types of frozen TMAs available at out institute: “Multi-tumor TMA” with >800 specimens from >50 tumor types including 60 prostate cancers and “normal tissue TMA” with >300 specimens from 32 tissue types.

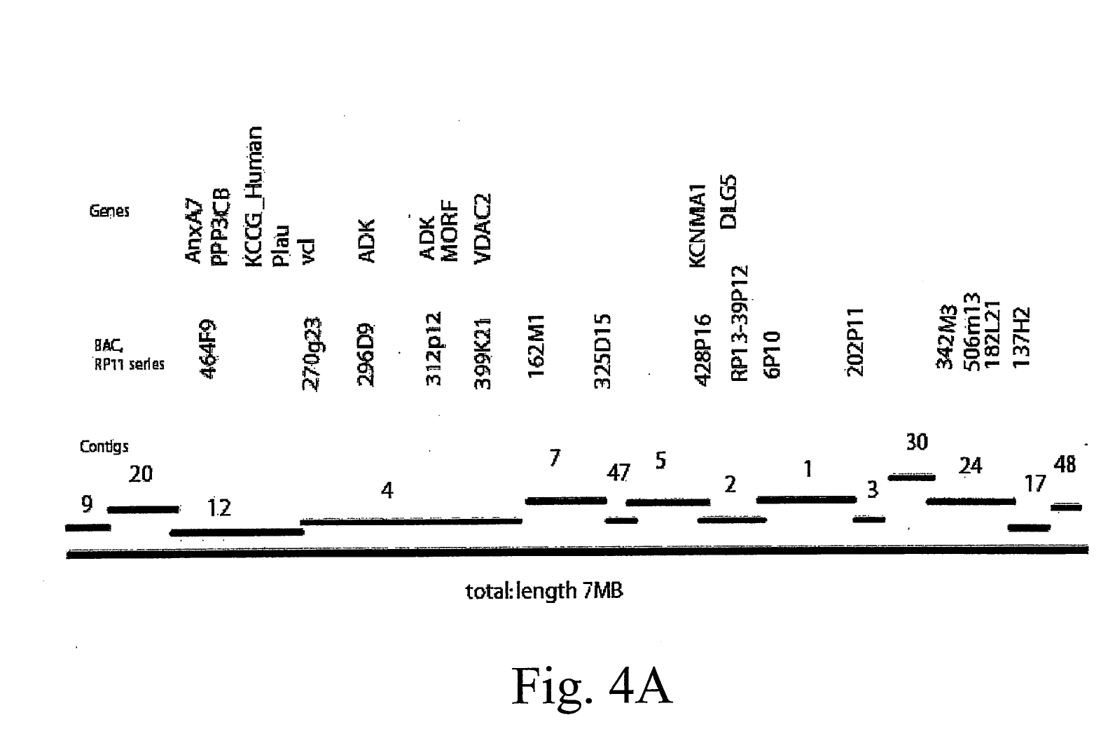

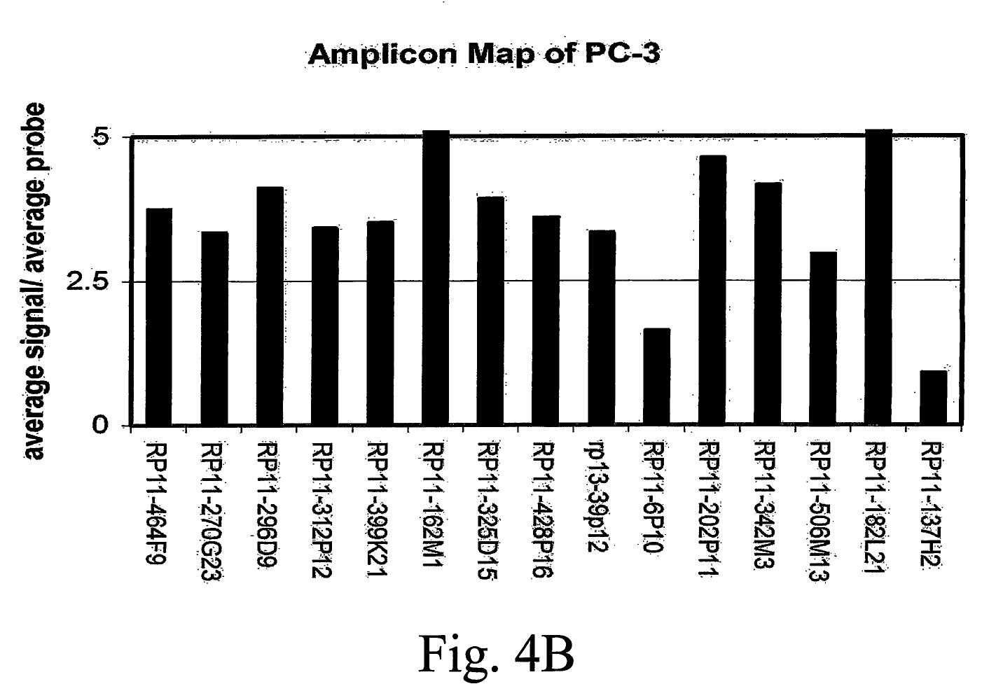

In a study that was supported by Swissbridge/Stammbach Stiftung, the present inventors explored the amplified DNA region at 10q22 in PC-3. A 7 Mb BAC contig across the amplified 10q22 region was analyzed by FISH on PC-3. FISH revealed an almost constant level of amplification across the region with no distinct amplification peak, suggesting that this amplification may not select a single target but rather be driven by several targets (FIG. 4).

The following series of genes was then selected for further analysis based on known or putative tumor-enhancing properties: Annexin 7, calcineurin 3, uPA (also called PLAU), KCNMA1, Vinculin (VCL), and DLG5. All of these genes were amplified by FISH. Interestingly, also the bladder cancer cell line JCA-1, which had initially been mistaken for a prostate cancer cell line, shows amplification at 10q22 by CGH (Pan Y, Lui WO, Nupponen N, et al. 5q11, 8p11, and 10q22 are recurrent chromosomal breakpoints in prostate cancer cell lines. Genes Chromosomes Cancer 2001; 30:187-195; and van Bokhoven A, Varella-Garcia M, Korch C, Miller G J. TSU-Pr1 and JCA-1 cells are derivatives of T24 bladder carcinoma cells and are not of prostatic origin. Cancer Res 2001; 61:63404). However, we found neither amplification by FISH nor increased expression of KCNMA1 in JCA-1. PTEN, a tumor-suppressor gene at 10q23.31 known to be inactivated in PC-3 was included as a control.

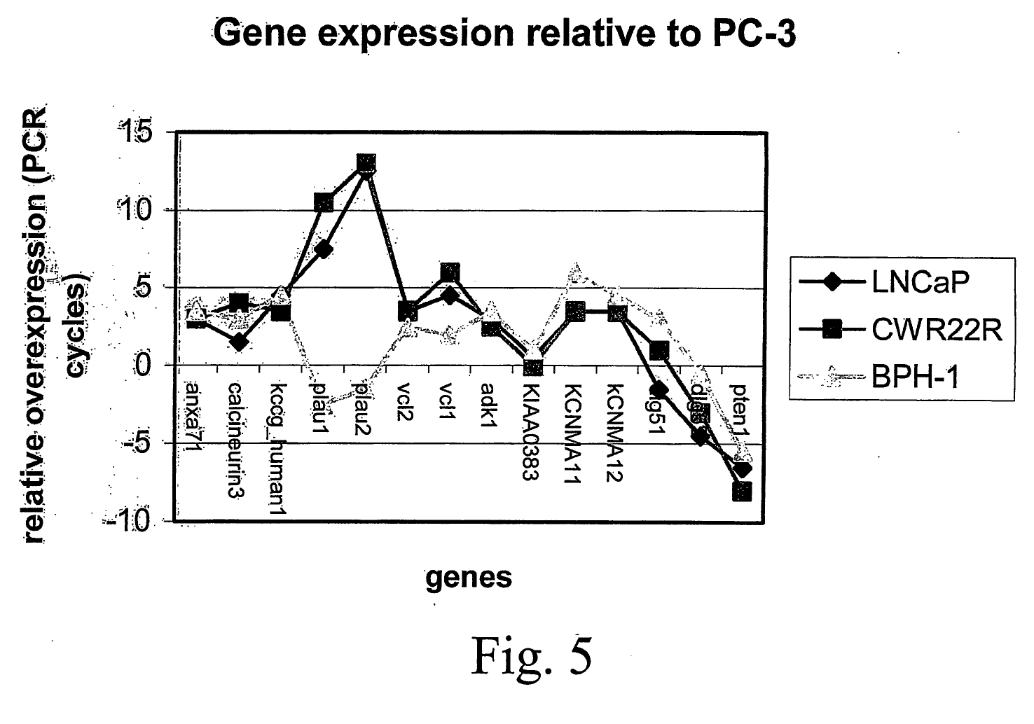

Quantitative real-time PCR using the LightCycler instrument (Roche, Mannheim, Germany) was applied to analyze the association between gene dosage and overexpression (FIG. 5). PC-3 was compared to 3 prostate cell lines without amplification at 10q22 including the prostate cancer cell lines LNCAP and CWR22R, and the benign prostate cell line BPH-1 which originates from benign prostatic hyperplasia. LightCycler analysis using 1-2 primer pairs per gene showed consistent over-expression of KCNMA1 in PC-3 as compared to the control cell lines. In contrast, expression of PLAU was higher in BPH-1 than in PC-3, putting a question mark to its previously suggested role as a prominent amplification target (Helenius M A, Saramaki O R, Linja M J, Tammela T L, Visakorpi T. Amplification of urokinase gene in prostate cancer. Cancer Res 2001; 61:5340-4). As expected, PTEN was downregulated in PC-3 as compared to the control cell lines (Vlietstra R J, van Alewijk D C, Hermans K G, van Steenbrugge G J, Trapman J. Frequent in-activation of PTEN in prostate cancer cell lines and xenografts. Cancer Res 1998; 58:2720-3).

KCNMA1 was then chosen for detailed analysis for the following reasons:
Consistent association between amplification and overexpression in the cell line model
Increasing evidence for oncogenic potential of potassium channels
Reported interaction with 17-β-estradiol and s-src.
Availability of a specific blocker for functional experiments (iberiotoxin)
   Analysis of KCNMA1 as a Therapeutic Target

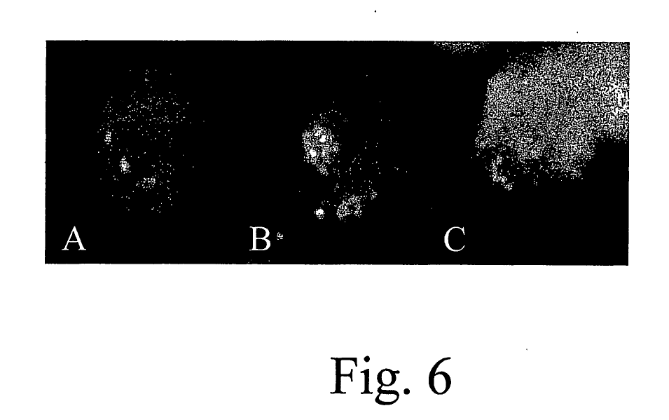

To be certain that KCNMA1 amplification is not only present in the PC-3 cell line but also in vivo, the inventors analyzed the prevalence of KCNMA1 amplification in clinical tumors by FISH on a prostate progression TMA with 535 prostate specimens. The FISH probe for KCNMA1 was generated from the genomic BAC clone rp11-428p16 (ID: AL731556, 203,003 bp). KCNMA1 amplification, defined as a gene/centromer 10 ratio of at least 2.5, was found in 13.5% of 141 locally recurrent or metastatic hormone-refractory tumors, but never in untreated early-stage tumors (FIG. 6).

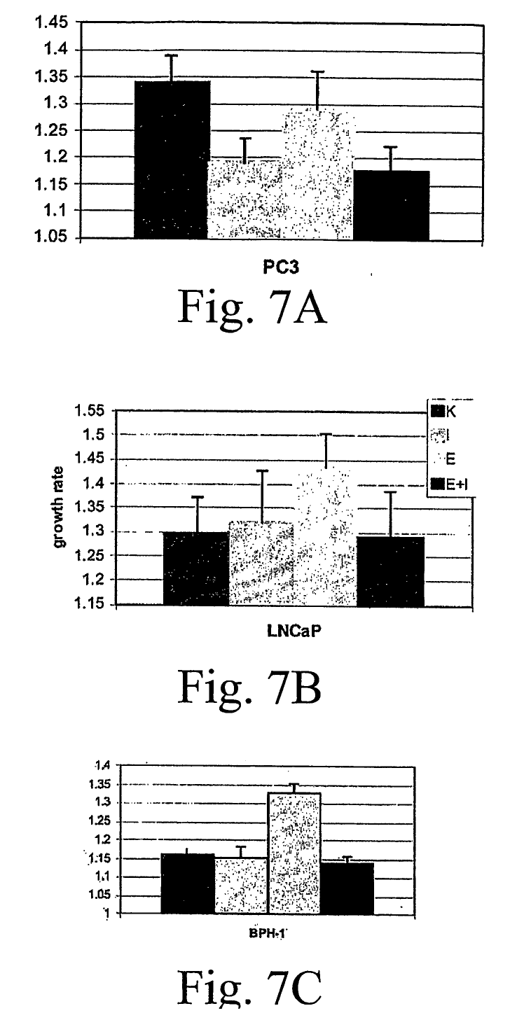

Next, the inventors explored the functional relevance of KCNMA1 amplification in the cell line models. Specific inhibition of KCNMA1 by iberiotoxin lead to a significant growth inhibition of PC-3 but had no significant effect on BPH-1 or LNCAP (FIG. 7). Interestingly, estradiol had a growth promoting effect on BPH-1 and LNCaP but not on PC-3. The effect of estradiol in BPH-1 and LNCaP was completely reversed by iberiotoxin, suggesting that the growth enhancement of BPH-1 and LNCaP by estradiol is mediated by KCNMA1.

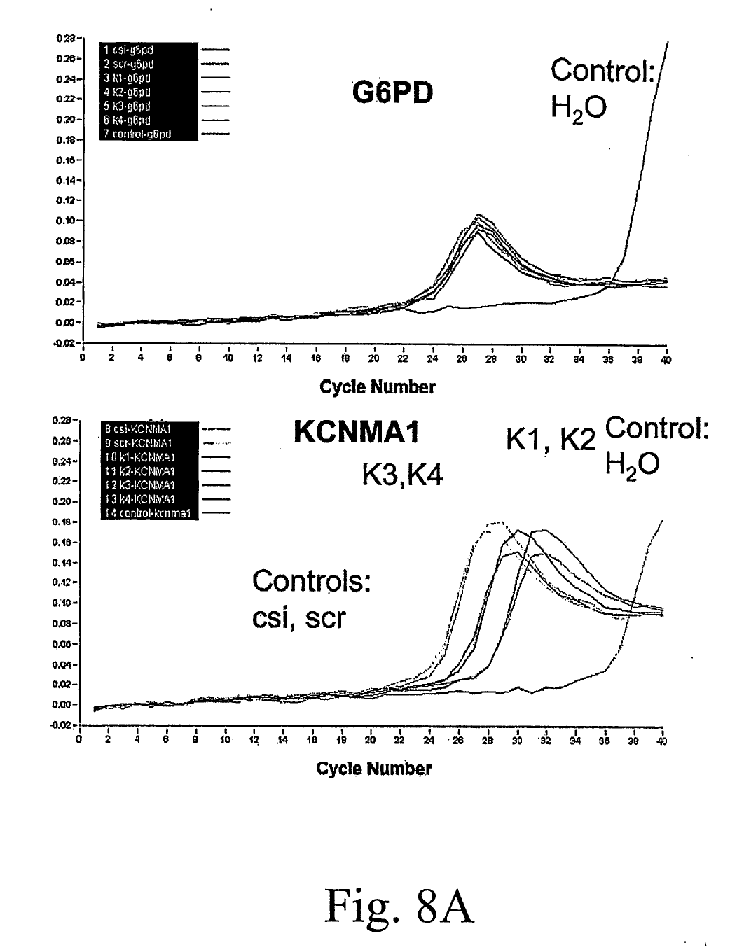

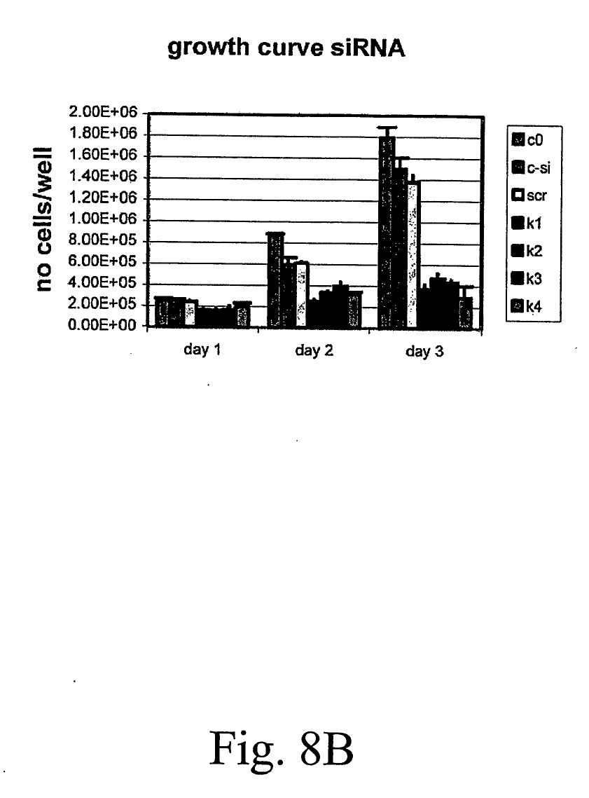

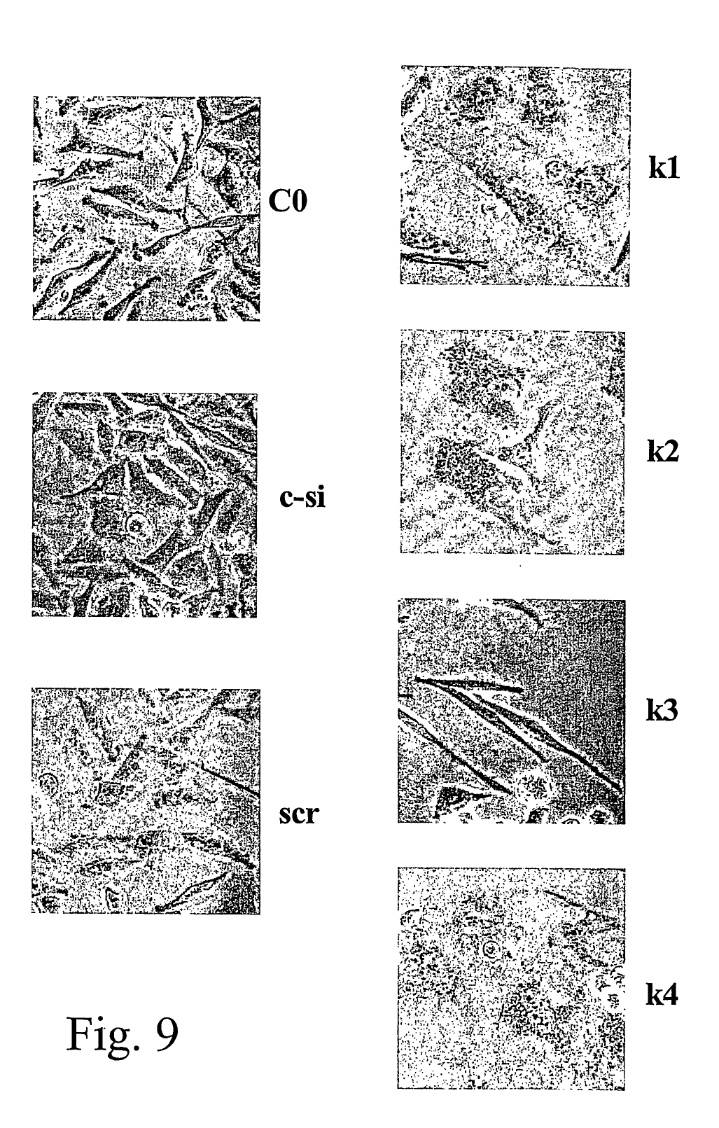

To further support these data and confirm the specific effect of KCNMA1 blockage in PC-3, the inventors established RNA interference (RNAi) technology for specific RNA inhibition. RNAi is a gene-specific mechanism for post-transcriptional gene silencing (PTGS) induced by double stranded RNA (Shi Y. Mammalian RNAi for the masses. Trends Genet 2003; 19:9-12; Wall N R, Shi Y. Small RNA: can RNA interference be exploited for therapy? Lancet 2003; 362:1401-3; and Zamore P D. Ancient pathways programmed by small RNAs. Science 2002; 296:1265-9). RNAi prevents the expression of a specific gene by disrupting the MRNA before it is translated to active protein. RNAi has become a powerful tool to obtain information about the function of specific genes in a quick and comparatively inexpensive manner. For this purpose, a short double stranded RNA (siRNA) is transfected into a human cell, where it causes degradation of RNAs containing homologous sequences. Specific blockage of KCNMA1 by siRNAs revealed a significant reduction of the growth rate of PC-3 (FIG. 8). This effect was demonstrated for four different KCNMA-1-specific siRNAs (K1-K4). In addition to this growth reduction, the inventors found a dramatic change of the cellular phenotype (FIG. 9). Three siRNAs (K1, K2 and K4) caused a spreading of the cells, suggesting changes of adhesion properties and/or cytoplasmic volume. Interestingly, the fourth siRNA (K3) resulted in an elongated and thin cellular shape. Taken together, our data suggest that the BK channel is not only involved in regulating growth but also in regulating cellular shape.

### EXAMPLE 1

### Analysis of KCNMA1 in Cell Lines

Materials and Methods

FISH: The genomic clones for KCNMA1 (rp11-428p16; ID: AL731556,203,003 bp) and uPA (rp11-417011; ID: AL596247, 228,061 bp) were obtained from the Sanger center, Cambridge, UK. The full sequences for these genomic BAC clones are lengthy, but they are publicly available from the U.S. National Institute of Health nucleotide sequence database at www.ncbi.nlm.nih.gov/entrez/query.fcgi?db=Nucleotide, and are incorporated herein by reference. The Bacteria were grown in LB containing chloramphenicol (Sigma). The DNA was extracted using the alkaline lysis miniprep protocol. The DNA was labeled using the Bionick kit (Invitrogen) replacing the Biotin by Digoxigenin (Roche). The probes were visualized with indirect immunofluorescence with FITC coupled antibodies. A commercially available centromeric probe of chromosome10 (Vysis) was used as a reference.

Lightcycler analysis of gene expression: The RNA was extracted from growing cells using trizol (Invitrogen) followed by DNA digestion using the RNA minikit (Qiagen). cDNA was synthesized with the superscript reverse transcriptase (Qiagen). The RT PCR was performed following supplier's suggestion (sybr green, Roche). Two different PCR primer pairs per gene were used (5′-3′): KCNMA1 paired primers, which included KCNMA1f/KCNMA1r and KCNMA2f/KCNMA2r, where KCNMA1f: ATATCCGCCCAGACACTGAC, KCNMA1r: ATCGTTGGCTGCAATAAACC, KCNMA2f: TTGGACCAAGACGATGATGA, KCNMA2r: CCTCTAAGGGCGTTTTCCTC; and uPA/plau paired primers, which included Plau1f/Plau1r and Plau2f/Plau2r, where Plau1f: ACTCCAAAGGCAGCAATGAA, Plau1r: GGCCTTTCCTCGGTAAAAGT, Plau2f: GTCACCACCAAAATGCTGTG, Plau2R: GCGGATCCAGGGTAAGAAGT.

Cell culture: As standard, the cells were grown in OPTIMEM (Invitrogen) containing 10% FCS (Amimed) and 1% Pen/Strep (Amimed). For iberiotoxin/17β-estradiol treatment 25000 cells were seeded in OPTIMEM, 10% FCS into 6 well plates (Falcon). After one day incubation, the medium was changed to OPTIMEM 1%FCS, 1% Pen/Strep. After another day of incubation the experiment was started by changing the Medium to OPTIMEM 0.4% Albumax (Invitrogen), 1% Pen/Strep containing either only solvents, 50 nM iberiotoxin (Sigma), 100 nM 17β-estradiol (Sigma), or a combination of both. After two days of incubation, the cells were collected and counted.

Results

After the mapping of the known amplicon to the genome and selection of potential target genes and bacterial artificial chromosomes (BACs) containing the relative sequences, a FISH probe of the Bac RP11-428p16 covering the gene KCNMA1 was made and hybridized to a tissue microarray (TMA) containing samples from a total of 141 locally recurrent or metastatic hormone-refractory tumors of which 13.5% (19 cases) showed KCNMA1 amplification (FIG. 6). Then, the expression of KCNMA1 on RNA level of four prostate cancer cell lines of interest (PC-3, LNCAP, CWR22R, BPH-1) (Table 2) was assayed using RT-PCR (lightcycler device, Roche). This analysis revealed that the amplified cell line clearly overexpresses KCNMA1 mRNA in relation to the not amplified cell lines LNCAP, CWR22R, and BPH-1 (FIG. 5).

TABLE 2
Androgen dependency and 10q22 amplification in prostate cell lines

| Cell Line | Amplification | Androgen Dependency |
|---|---|---|
| PC-3 | + | − |
| LNCap | − | + |
| CWR22R | − | − |
| BPH-1 | − | + |

This suggests that the amplification at 10q22 could be a mechanism to increase KCNMA1 expression. Knowing that slowpoke, the protein encoded by KCNMA1, is activated by 17β-estradiol and inhibited by iberiotoxin and taking into account that potassium ions are suspected to influence proliferation or apoptosis we performed a simple functional experiment testing the response in growth rate of the mentioned cell lines on treatment with 17β-estradiol and/or iberiotoxin. The results showed that the growth rate of the amplified and androgen independent cell line PC-3 could be reduced with iberiotoxin (FIG. 7). In contrast, LNCAP did not react on iberiotoxin addition but increased its growth rate upon 17β-estradiol addition. This effect could be blocked by adding iberiotoxin to the 17β-estradiol treated cells (FIG. 7).

### EXAMPLE 2

### RNA Interference

Transfection

The siRNA was obtained from Qiagen. The siRNa were handled as suggested: they were diluted in 1 ml of the provided buffer (100 mM potassium acetate, 30 mM HEPES-KOH, 2 mM magnesium acetate, pH=7.4). The solution was incubated for 1 min at 90° C., then it was incubated one hour at 37° C. to dissolve the siRNA. This solution was directly used as stock solution in our experiments.

siRNA target sequences:

| | |
|---|---|
| k1: | gactggcagagtcctggttgt |
| k2: | gtgggtctgtccttccctact |
| k3: | gaccgtcctgagtggccatgt |
| k4: | acgcccttagaggtggctaca |

We transfected the cells (only the cell line PC-3) using Lipofectamine 2000 (Invitrogen) following the provided protocol. In brief 50,000 cells were plated per well of a 6 well plate (Falcon) in 3 ml Optimen Invitrogen) +10% FCS and grown for one day. The transfection complexes were prepared by carefully mixing 250 □l Optimem and 5 μl Lipofectamine, and 250 μl Optimem and 5 μl siRNA, respectively. After 5 min incubation the two solutions were mixed and incubated 20 min at room temperature. The cells were washed with Optimem once and then 2.5 ml Optimem was added. Then 500 μl or the transfection complexes was added. The cells were incubated for 4 h at 37° C. at standard growing conditions (37° C., 5% CO₂). Then the transfection complexes were washed off and 3 ml Optimem+10% FCS was added.

mRNA Expression Analysis:

RNA was prepared one day after transfection using the RNAeasy minikit (Invitrogen) following the suggested protocol. Then 0.5 μg RNA was transcribed into 1° strand cDNA using the Superscript First Strand Synthesis System for RT PCR kit (Invitrogen) using oligo dT as primers. Then the relative expression was measured in a LightCycler device (Roche) using Hybridization probes. Both, KCNMA1 and G6PD primers and probes were designed and synthesized by TIB MolBiol.

PCR Primer: KCNMA1

| | |
|---|---|
| KCNMA1a: | TTCTgggCCTCCTTCgTCT |
| KCNMA1s: | CCTggCCTCCTCCATggT |

G6PD:

| | |
|---|---|
| G6ex6 S: | ACCACTACCTgggCAAggAg |
| G6ex7, 8 R: | TTCTgCATCACgTCCCggA |

Hybridization probes: KCNMA1:

AgCgTCCgCCAgAgCAAgAT X
ATgAAgAggCCCCCgAAgAAAgT p
G6PD: CAgATggggCCgAAgATCCTgTT FL
CAAATCTCAgCACCATgAggTTCTgCAC PH

The PCR was performed using the Light cycler Fast Start DNA Master Hybridization Probes kit (Roche) following the contained protocol.
Growth Curve

For this assay we transfected PC-3 cells as described above with the active siRNA K1-4. As controls we didn't transfect the cells at all (c0), added no RNA to the transfection mix (c-si) or transfected inactive scrambled sequence control siRNA (Qiagen) named scr in the FIG. 8 of the grant application.

The siRNA transfected cells were grown for 1, 2, or 3 days in Optimem +10% FCS. Then they were trypsinzed in 1 ml Trypsin solution (Amimed) and counted the cell number using a Neubauer chamber. This assay was done three times. We also looked at the cell morphology at day 1-4 after transfection.

Results

The results of the siRNA experiments were as described above.

### EXAMPLE 3

### Electrophysiological Analysis

Patch clamp recording was used to further assess the role of KCNMA1.

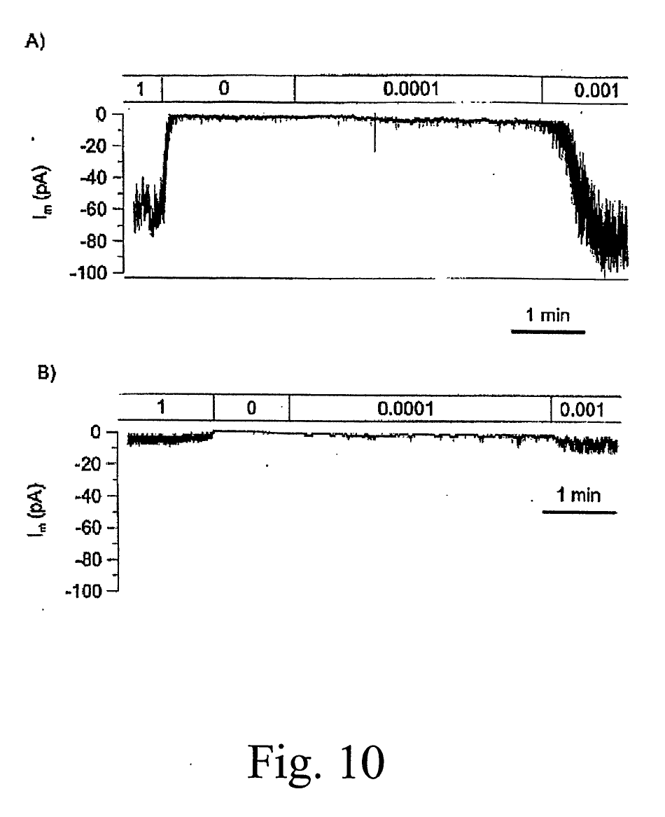

In preliminary whole cell patch clamp experiments we found a clear inhibitory effect of iberiotoxin (30 nM) on whole cell conductances of PC-3 cells, while no significant effects were detected in BPH-1 cells. The membrane voltage of PC-3 cells is hyperpolarized when compared with that of BPH-1 cells. Moreover, paxillin inhibited potassium channels in cell excised inside/out membrane patches of PC-3 cells, but not in membrane patches of BPH-1 cells. In cell excised inside/out membrane patches of PC-3 cells we found pronounced activity of Ca²⁺ activated K⁺ channels, while little channel activity was found in excised membrane patches of BPH-1 cells (FIG. 10). Removal of Ca²⁺ from the cytosolic side (0 mmol/l) abolished channel activity in both membrane patches of PC-3 and BPH-1 cells. Channel activity was recovered at cytosolic Ca²⁺ concentrations of 1 μmol/l (0.001). These preliminary results fit very well to the expression data of KCNMA1 obtained so far.

While the present invention has been described with reference to certain preferred embodiments, one of ordinary skill in the art will recognize that additions, deletions, substitutions, modifications and improvements can be made while remaining within the spirit and scope of the present invention as defined by the appended claims.

1. A method for detecting the expression level of KCNMA1, and optionally Urokinase (uPA), in a tissue sample, the method comprising:
providing one or more probes and a tissue sample, the tissue sample including nucleotides;
hybridizing the one or more probes to the nucleotides in the tissue sample to produce hybridization results; and
determining an expression level for the KCNMA1 gene, and, optionally for the Urokinase (uPA) gene, from the hybridization results.

2. A method as recited in claim 1, wherein said hybridization comprises in situ hybridization.

3. A method as recited in claim 1, wherein said hybridization occurs in a tissue microarray.

4. A method as recited in claim 1, further comprising amplifying the KCNMA1 gene, and optionally the Urokinase (uPA) gene from the tissue to produce amplified DNA.

5. A method as recited in claim 1, further comprising identifying a level of expression of KCNMA1 mRNA, and, optionally, the Urokinase (uPA) gene, by using a primer selected from the group consisting of: KCNMA1f, KCNMA1r, and KCNMA1 probes.

6. A method as recited in claim 1, wherein the tissue sample collected is a prostate tumor tissue sample.

7. A method as recited in claim 1, further comprising identifying a KCNMA1 mRNA, and optionally a Urokinase (uPA) mRNA, by using one or more PCR primers selected from the group consisting of KCNMA1f:

| | | |
|---|---|---|
| ATATCCGCCCAGACACTGAC, | (SEQ ID No. 1) | KCNMA1r: |
| ATCGTTGGCTGCAATAAACC, | (SEQ ID No. 2) | KCNMA2f: |
| TTGGACCAAGACGATGATGA, | (SEQ ID No. 3) | KCNMA2r: |
| CCTCTAAGGGCGTTTTCCTC, | (SEQ ID No. 4) | Plau1f: |
| ACTCCAAAGGCAGCAATGAA, | (SEQ ID No. 5) | Plau1r: |
| GGCCTTTCCTCGGTAAAAGT, | (SEQ ID No. 6) | Plau2f: |
| GTCACCACCAAAATGCTGTG, | (SEQ ID No. 7) | and Plau2R: |
| GCGGATCCAGGGTAAGAAGT. | (SEQ ID No. 8) | |

8. A method for treating a patient with cancer comprising:
collecting a tumor tissue sample;
analyzing the tumor tissue sample to determine an expression level of KCNMA1, and optionally Urokinase (uPA), to determine whether KCNMA1 overexpression is absent or present and whether Urokinase (uPA) overexpression is present or absent; and
when KCNMA1 overexpression or Urokinase (uPA) overexpression is present, treating the patient with an anti-tumor agent.

9. A method as recited in claim 8, wherein the anti-tumor agent is a blocking agent of potassium channels.

10. A method as recited in claim 9, wherein the anti-tumor agent is an iberiotoxin.

11. A method as recited in claim 8, wherein the patient is treated with the anti-tumor agent when both KCNMA1 gene amplification and Urokinase (uPA) gene amplification are present.

12. A method as recited in claim 11, wherein the anti-tumor agent is a blocking agent of potassium channels.

13. A method as recited in claim 12, wherein the anti-tumor agent is an iberiotoxin.

14. A method as recited in claim 8, further comprising identifying the KCNMA1 gene, and optionally the Urokinase (uPA) gene, by using one or more PCR primers selected from the group consisting of KCNMA1f:

| | | |
|---|---|---|
| ATATCCGCCCAGACACTGAC, | (SEQ ID No. 1) | KCNMA1r: |
| ATCGTTGGCTGCAATAAACC, | (SEQ ID No. 2) | KCNMA2f: |
| TTGGACCAAGACGATGATGA, | (SEQ ID No. 3) | KCNMA2r: |
| CCTCTAAGGGCGTTTTCCTC, | (SEQ ID No. 4) | Plau1f: |
| ACTCCAAAGGCAGCAATGAA, | (SEQ ID No. 5) | Plau1r: |
| GGCCTTTCCTCGGTAAAAGT, | (SEQ ID No. 6) | Plau2f: |
| GTCACCACCAAAATGCTGTG, | (SEQ ID No. 7) | and Plau2R: |
| GCGGATCCAGGGTAAGAAGT. | (SEQ ID No. 8) | |

15. A method as recited in claim 8, wherein said cancer is prostate cancer.

16. A method as recited in claim 15, wherein said patient has developed, is developing, or is suspected to have developed hormone-refractory prostate cancer.

17. A method of identifying a drug for treating cancer, comprising the steps of:
providing a first cell line expressing a large-conductance, Ca²⁺-activated potassium channel;
providing a second cell line which expresses the channel at a lower level than the first cell line, or does not express the channel at all;
providing a candidate compound for treating cancer;
incubating the candidate compound with the cell lines to produce a response in each cell line;
comparing the response of the first cell line to the response of the second cell line.

18. A method as recited in claim 17, wherein said candidate compound comprises a compound known or suspected to block a potassium channel.

19. A method as recited in claim 17, wherein said response is a rate of growth of the cell line.

20. A method as recited in claim 17, wherein said channel is KCNMA1.

21. A compound identified by the method of claim 17.
